(19) United States
(12) Patent Application Publication
(10) Pub. No.: US 2012/0323143 A1
(43) Pub. Date: Dec. 20, 2012

(54) FLUID COLLECTION/INJECTION DEVICE UTILIZING A STANDARD INTERFACE NEEDLE, NEEDLE ASSEMBLY FOR THE DEVICE, AND METHOD USING THE SAME

(75) Inventor: Steven Schraga, Surfside, FL (US)

(73) Assignee: STAT MEDICAL DEVICES, INC., North Miami, FL (US)

(21) Appl. No.: 13/524,742

(22) Filed: Jun. 15, 2012

Related U.S. Application Data
(60) Provisional application No. 61/498,133, filed on Jun. 17, 2011.

(51) Int. Cl. A61M 5/34; A61M 5/50; A61B 5/15

(52) U.S. Cl. 600/576; 604/240; 604/241; 604/110

(57) ABSTRACT

Fluid collection device includes a body having a front end and a back end. The back end is open and/or sized and configured to receive therein a receptacle. A needle holding member is arranged in an area of the front end of the body. A needle holding member comprising a needle connecting interface adapted to mate with a needle interface

### CROSS-REFERENCE TO RELATED APPLICATIONS

The instant application is a U.S. non-provisional Application that is based on and claims the benefit of U.S. provisional application No. 61/498,133, filed Jun. 17, 2011, the disclosure of which is hereby expressly incorporated by reference thereto in its entirety.

### BACKGROUND OF THE INVENTION

1. Field of the Invention

This invention relates generally to devices used to collect fluid samples from and/or inject fluids into patients. More specifically, this invention relates to a device which utilizes a holder utilizing a double-ended needle that can be released, retracted or removed from the holder in a more safe and easy manner. The device can be a single-use device or multi-use device. The invention also relates to a method of collecting a fluid sample with the device as well as a method of making the device. The invention also relates to a blood sample collection device that is easier to use, is less costly to produce, is safer to use, and/or is easier to manufacture.

2. Discussion of Background Information

Prevention of needle sticks is of paramount concern in the healthcare industry because of serious and deadly risk factors associated with AIDS and other serious communicable diseases. Typical blood collection devices utilize a needle inserted into a patient's vein so as to draw blood through the needle into an associated separate collection reservoir. Accidental needle sticks from previously used needles can occur during the fluid withdrawing process and subsequent handling and disposal operation. Until such used medical devices are destroyed, they remain a risk to those handling them.

Devices used for blood sampling are well know and include a collection device sold under the trademark Vacutainer® by Becton Dickinson Corporation. This device has a tubular syringe-like body with a needle in the front end, part of which extends back into a tubular syringe-like shell. Part of the needle extends externally for punching the skin. An evacuated collection tube with a rubber stopper is placed into the open back of the syringe-like shell with the rubber stopper against the internal end of the needle. After the skin is punctured, the collection tube is pushed forward to cause the needle to enter the evacuated tube. Vacuum helps draw blood into the collecting tube. When a sufficient sample has been obtained, the collecting tube and the stopper are simply withdrawn from the tubular shell and sent to the laboratory. This particular device has a permanently extended needle and an opening in the back for the collection tube which remains open after the collection tube is removed, leaving small quantities of blood and an internally exposed needle.

Medical devices which are used for collecting fluid samples from patients which have quick release needle systems are also known. Such devices include: U.S. Pat. No. 5,797,490 to FUJI et al; U.S. Pat. No. 5,755,673 to KINSEY; U.S. Pat. No. 4,822,343 to BEISER; U.S. Pat. No. 4,984,580 WANAMAKER; U.S. Re. 38,964 to SHILLINGTON; U.S. Pat. No. 5,616,136 to SHILLINGTON et al.; U.S. Pat. No. 5,637,101 to SHILLINGTON; U.S. Pat. No. 5,117,837 to WANAMAKER et al.; U.S. Pat. No. 4,907,600 to SPENCER; U.S. Pat. No. 4,993,426 to SPENCER; U.S. Pat. No. 4,904,244 to HARSH et al.; U.S. Pat. No. 4,490,142 to SILVERN. The disclosures of each of these documents is expressly incorporated by reference herein in their entireties.

Medical devices which are used for collecting fluid samples from patients which can benefit from the improvement offered by the instant invention include: U.S. 2010/0286558 to SCHRAGA; U.S. 2008/0262421 to SCHRAGA; U.S. Ser. No. 12/974,908 to SCHRAGA filed on Dec. 21, 2010; and U.S. 61/480,787 to SCHRAGA filed on Apr. 29, 2011. The disclosures of each of these documents is expressly incorporated by reference herein in their entireties.

The invention aims to improve devices of the type described above by making a needle assembly for a fluid collection device which includes a standard interface needle. The device is also believed to be as safe or safer to use and/or dispose-of than the above-noted devices.

### SUMMARY OF THE INVENTION

According to one non-limiting aspect of the invention there is provided a fluid collection/injection device including one or more features shown herein in combination with one or more devices disclosed above. In embodiments, there is provided a fluid collection/injection device including one or more features shown herein in combination with one or more devices disclosed in U.S. 2010/0286558 to SCHRAGA; U.S. 2008/0262421 to SCHRAGA; U.S. Ser. No. 12/974,908 to SCHRAGA filed on Dec. 21, 2010; and U.S. 61/480,787 to SCHRAGA filed on Apr. 29, 2011. The disclosures of each of these documents is expressly incorporated by reference herein in their entireties.

The invention provide for a fluid collection device comprising a body having a front end and a back end. The back end that is at least one of open and sized and configured to receive therein a receptacle. A needle holding member is arranged in an area of the front end of the body. A needle holding member comprising a needle connecting interface adapted to mate with a needle interface.

The needle holding member may be a spring biased member that is movable from an initial position to a retracted position within the body.

The needle connecting interface may be a luer-lock interface, a standard interface and/or a universal interface.

The fluid collection device may further comprise a spring structured and arranged to move the needle holding member to a retracted position within the body when a movable member arranged with the body experiences at least one of: a front end of the movable member is caused to expand generally radially; a back end of the movable member is caused to contract generally radially; a front end of the movable member is caused to expand generally radially when a back end of the movable member is caused to contract generally radially; axial movement caused by a cap closing off the back end of the body, wherein, during the closing off of the back end of the body, a tapered surface of a back end of the movable member is contacted by a portion of the cap; circumferential portions of a front end of the movable member are caused to expand outwardly or generally radially so as to allow the needle holding member to disengage from the front end of the movable member; and portions of a front end of the movable member separated by slots are caused to expand outwardly or generally radially so as to allow the needle holding member to disengage from the front end of the movable member.

The invention also provides for a fluid collection/injection device comprising a body having a front end, a back end, and a main hollow section arranged between the front and back ends. A needle hub securing section is arranged on the front end and being structured and arranged to receive therein a needle member. The needle hub securing section comprises a fixed part and a movable part. A needle holding member is connectable to the needle hub securing section. The needle holding member comprising a needle connecting interface adapted to mate with a needle interface. The fixed part is integrally formed with the front end and the movable part is arranged on a member that has one end which is one of: fixed to a portion of the main hollow section; connected to a portion of the main hollow section via a living hinge; removably connected to a portion of the main hollow section; and integrally formed with the main hollow section.

The body may be one of generally cylindrical and generally tubular.

The fixed part and the movable part may form a generally cylindrical section which the movable part is in an initial position.

The fixed part and the movable part may each generally comprise one-half of an internal locking thread structured and arranged to engage with an external thread of the needle member.

The member may have one end which is fixed to the portion of the main hollow section.

The member may have one end which is removably connected to the portion of the main hollow section.

The invention also provides for a retractable medical device comprising a body having and a back end that is at least one of open and sized and configured to receive therein a receptacle, a movable member arranged within the body, the movable member having a back end and a front end, a movable holding member arranged in an area of the front end of the movable member, a needle connecting interface adapted to mate with a needle interface and being movable with the movable holding member, and a spring structured and arranged to move the movable holding member to a retracted position within the movable member when the movable member experiences at least one of: the front end of the movable member is caused to expand generally radially; the back end of the movable member is caused to contract generally radially; the front end of the movable member is caused to expand generally radially when the back end of the movable member is caused to contract generally radially; axial movement caused by a cap closing off the back end of the body, wherein, during the closing off of the back end of the body, a tapered surface of the back end of the movable member is contacted by a portion of the cap; circumferential portions of the front end of the movable member are caused to expand outwardly or generally radially so as to allow the needle holding member to disengage from the front end of the movable member; and portions of the front end of the movable member separated by slots are caused to expand outwardly or generally radially so as to allow the needle holding member to disengage from the front end of the movable member.

The needle connecting interface may be a luer-lock interface, a standard interface and/or a universal interface.

The invention also provides for a fluid collection/injection device comprising a body having a front end, a back end, and a main hollow section arranged between the front and back ends, a needle hub securing section arranged on the front end, a needle receiving member having a needle connecting interface, and the fluid collection/injection device is structured and arranged to utilize at least an operational mode, an operational mode, and a post-use mode, in the installation mode, the needle receiving member being coupled to the body via the needle hub securing section, in the operational mode, fluid passing through the needle receiving member and into or out of a receptacle inserted into the main hollow section; and in the post-use mode, a safety cover at least one of; prevents re-use of the fluid collection/injection device; prevents removal of the needle receiving member from the fluid collection/injection device; prevents removal of the needle receiving member from the needle hub securing section; locks in a covering position and prevents removal of a needle from the needle receiving member; activates release of the needle receiving member from the body such that when the safety cover is in a position covering a needle of the needle receiving member, the needle receiving member can fall out of the needle hub securing section; activates release of the needle receiving member from the body such that when the safety cover is in a position covering a needle of the needle receiving member, the needle receiving member is no longer securely retained to the needle hub securing section; releases a securing engagement between the needle receiving member and the needle hub securing section; unlocks a locking connection between the needle receiving member and the needle hub securing section; and moves a mechanism arranged in an area of the needle hub securing section which releases a connection between the needle receiving member and the needle hub securing section.

The needle connecting interface is a luer-lock interface, a standard interface and/or a universal interface.

The invention also provides for a method drawing fluid using the device of any of the types disclosed herein, wherein the method comprises installing the needle connecting interface and thereafter installing a needle having a standard interface on the needle connecting interface.

The invention also provides for a method drawing fluid using the device of any of the types disclosed herein, wherein the method comprises installing a needle having a standard interface on the needle connecting interface of the needle holding member so as to form a needle assembly and thereafter installing the needle assembly on the body.

The invention also provides for a method drawing fluid using the device of any of the types disclosed herein, wherein the method comprises installing a needle having a standard interface on the needle connecting interface and one of injecting fluid through the needle and withdrawing fluid into a container installed in the body via the needle.

The invention also provides for a needle assembly for a fluid collection device comprising a needle holding member installable on a fluid collection device, a needle connecting interface arranged on one end of the needle holding member, and a needle having a needle interface, wherein the needle is at least one of installable on the needle holding member such that the needle is in fluid communication with another needle arranged on the needle holding member and removably installable on the needle holding member.

The invention also provides for a double-ended needle assembly comprising a needle holding member installable on a fluid collection device and comprising a needle connecting interface arranged on one end of the needle holding member and a needle arranged on another end and a needle having a standard needle interface compatible with the needle connecting interface of the needle holding member.

The needle may be at least one of installable on the needle holding member such that the needle is in fluid communication with the needle arranged on the needle holding member and removably installable on the needle holding member.

Other exemplary embodiments and advantages of the present invention may be ascertained by reviewing the present disclosure and the accompanying drawing.

### BRIEF DESCRIPTION OF THE DRAWINGS

The present invention is further described in the detailed description which follows, in reference to the noted plurality of drawings by way of non-limiting examples of exemplary embodiments of the present invention, in which like reference numerals represent similar parts throughout the several views of the drawings, and wherein:

FIG. 1 shows a needle holding member in accordance with one non-limiting embodiment of the invention. The member is shown in a prior-use configuration such that an interface cover is shown arranged on the interface portion of the member;

FIG. 2 shows a needle member in accordance with one non-limiting embodiment of the invention. The needle member is shown in a prior-use configuration such that a needle cover is shown arranged on the needle portion of the needle member;

FIG. 3 shows the needle holding member of FIG. 1 in a ready to use configuration such that an interface cover is shown removed so as to expose the interface portion of the member;

FIG. 4 shows the needle holding member of FIG. 3 in a configuration wherein needle cover is shown retracted. This typically occurs only when the member is installed on a fluid collection device and a collection vial is fully inserted therein;

FIG. 5 shows a needle member similar to that shown in FIG. 2 in a ready to use configuration;

FIGS. 6 and 7 show different needle members from that of FIG. 5, but which utilize the same interface or connecting portion so as to be usable with the member of FIG. 1. In FIG. 6, the needle is different or shorter in length than that of FIG. 5. In FIG. 7, the needle is different or shorter in length and different or smaller in diameter than that of FIG. 5;

FIG. 8 shows the needle member of FIG. 5 as it is about to be connected to the needle holding member of FIG. 3. This can occur before the needle holding member is installed on a fluid collection device or after;

FIG. 9 shows a needle assembly resulting from the connection of the components shown in FIG. 8;

FIG. 10 shows the needle holding member of FIG. 3 installed on a fluid collection device in accordance with one non-limiting embodiment of the invention;

FIG. 11 shows the needle assembly of FIG. 9 installed on a fluid collection device in accordance with one non-limiting embodiment of the invention;

FIG. 12 shows a needle holding member in accordance with another non-limiting embodiment of the invention. The member is shown in a ready to use configuration such that an interface cover has been removed from the interface portion of the member;

FIG. 13 shows the needle holding member of FIG. 12 installed on a fluid collection device in accordance with another non-limiting embodiment of the invention;

FIG. 14 shows a needle assembly installed on a fluid collection device in accordance with another non-limiting embodiment of the invention;

FIG. 15 shows the device of FIG. 14 before the needle member is connected to the needle holding member already installed on the fluid collection device in accordance with another non-limiting embodiment of the invention;

FIG. 16 shows a needle holding member in accordance with another non-limiting embodiment of the invention. The member is shown in a prior-use configuration such that an interface cover is shown arranged on the interface portion of the member;

FIG. 17 shows a needle member in accordance with another non-limiting embodiment of the invention. The needle member is shown in a prior-use configuration such that a needle cover is shown arranged on the needle portion of the needle member;

FIG. 18 shows a needle assembly in accordance with another non-limiting embodiment of the invention. In this embodiment, the needle member is already installed on the needle holding member, a needle cover is installed on the needle of the needle member, and a safety cover is pivotally connected to the needle holding member; and

FIG. 19 shows a needle holding member in accordance with another non-limiting embodiment of the invention. The member has two main components which are threadably connected to one another.

### DETAILED DESCRIPTION OF THE INVENTION

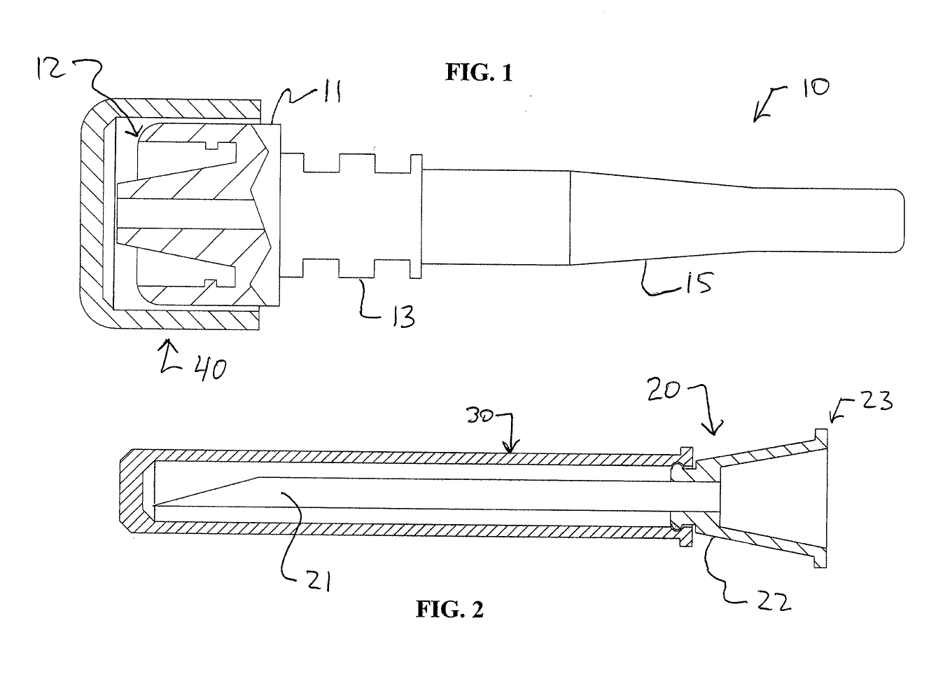

FIGS. 1-4 show a first non-limiting embodiment of the invention. In FIG. 1, a needle holding member 10 in accordance with one non-limiting embodiment of the invention has hub portion 11, a connecting interface portion 12, a retaining section 13, an inner hollow needle 14, and a retractable needle cover 15. The configuration of the interface 12, i.e., the receiving portion thereof, can be of any conventional standard type interface such as a luer-lok type interface. Similarly, the configuration of elements 13, 14, and 15 can be of any conventional type.

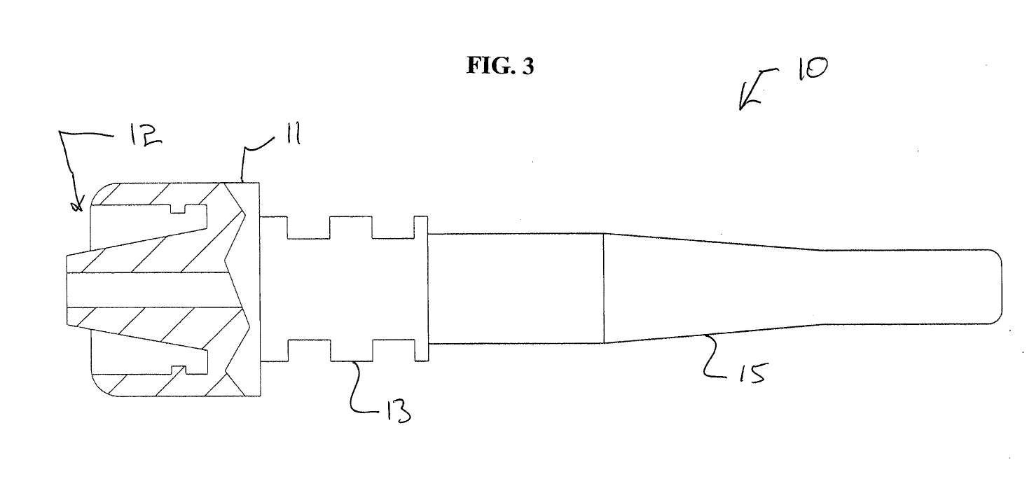

In FIG. 2, a needle member 20 in accordance with one non-limiting embodiment of the invention is shown in a prior-use configuration. In this configuration, a removable needle cover 30 is shown mounted covering the hollow needle portion 21 of the needle member 20. The cover 30 has a closed proximal end and an open distal end which is removably and releasably connected (via e.g., a snap and/or projection/recess connection) to the connecting interface section 23 of the member 20.

In FIG. 3, the needle holding member 10 of FIG. 1 is shown in a ready to use configuration such that an interface cover 40 (see FIG. 1) has been removed so as to expose the interface portion 12 of the member 10.

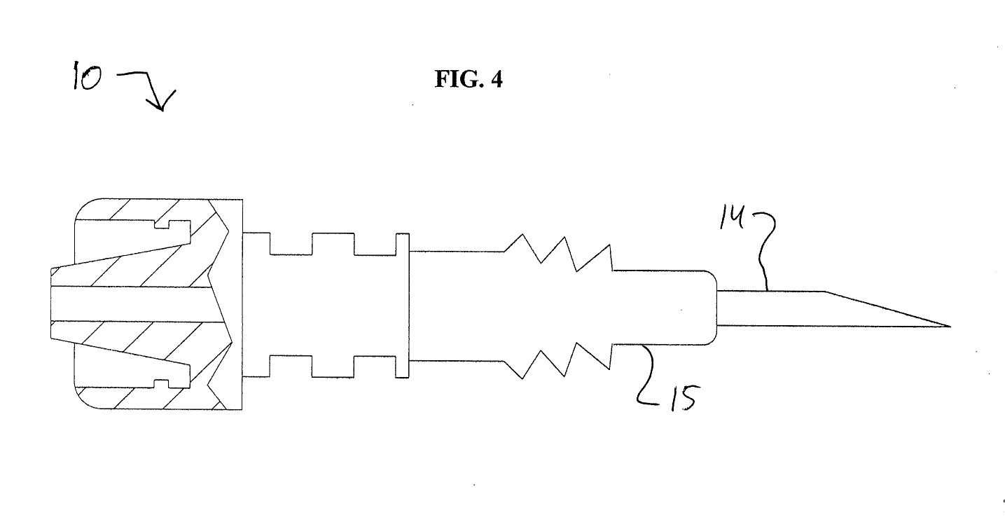

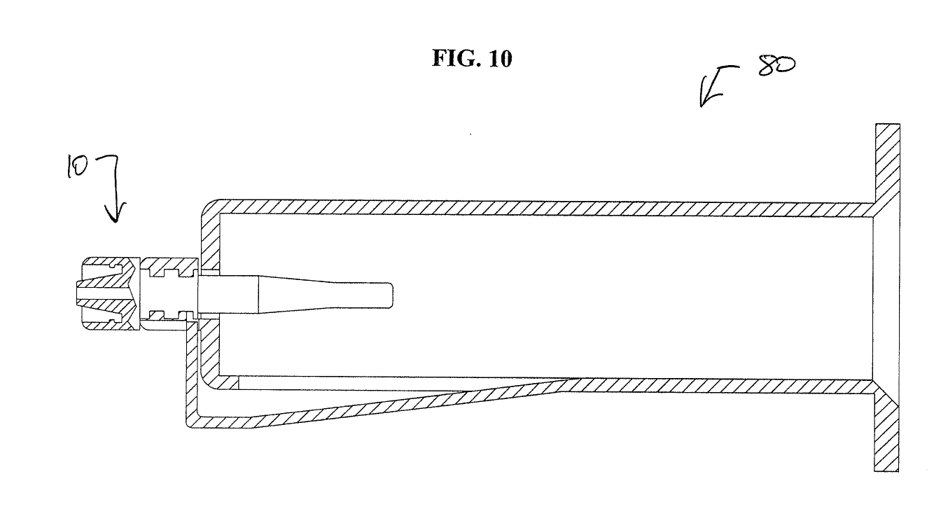

FIG. 4 shows the needle holding member 10 of FIG. 3 in a configuration wherein needle cover 15 is retracted to expose the inner needle 14. This typically occurs only when the member 10 is installed on a fluid collection device (see e.g., FIG. 10) and when a collection vial is fully inserted into the fluid collection device. A typical vacutainer-type vial has an insertion end whose self-sealing septum can be punctured by the needle 14 and can cause retraction of the flexible cover 15 during insertion of the vial.

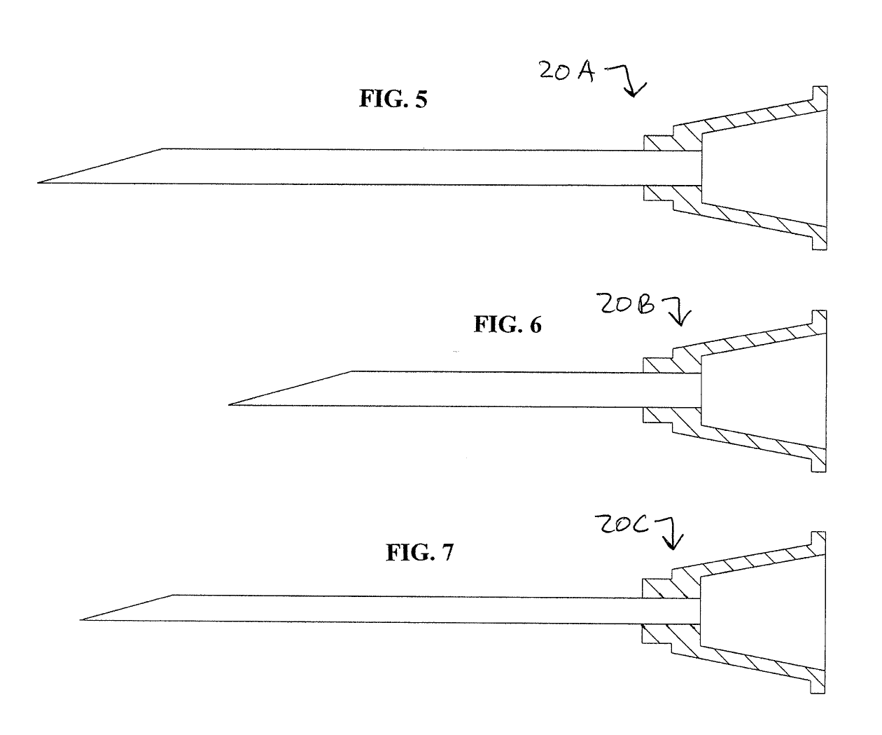

FIGS. 5-7 show how different sized needle members 20 having a common standard connecting interface can be used with the needle holding member 10 of FIG. 1. In FIG. 5, the needle portion of the member 20A has a diameter and an axial length that is larger than that of the member of FIG. 7. In FIG. 6, the needle portion of the member 20B has a diameter that is larger than that of the member of FIG. 7, but a length that is longer. In FIG. 7, the needle portion of the member 20C has a diameter that is smaller than that of the member 20A of FIG. 5, but a length that is between that of FIGS. 5 and 6.

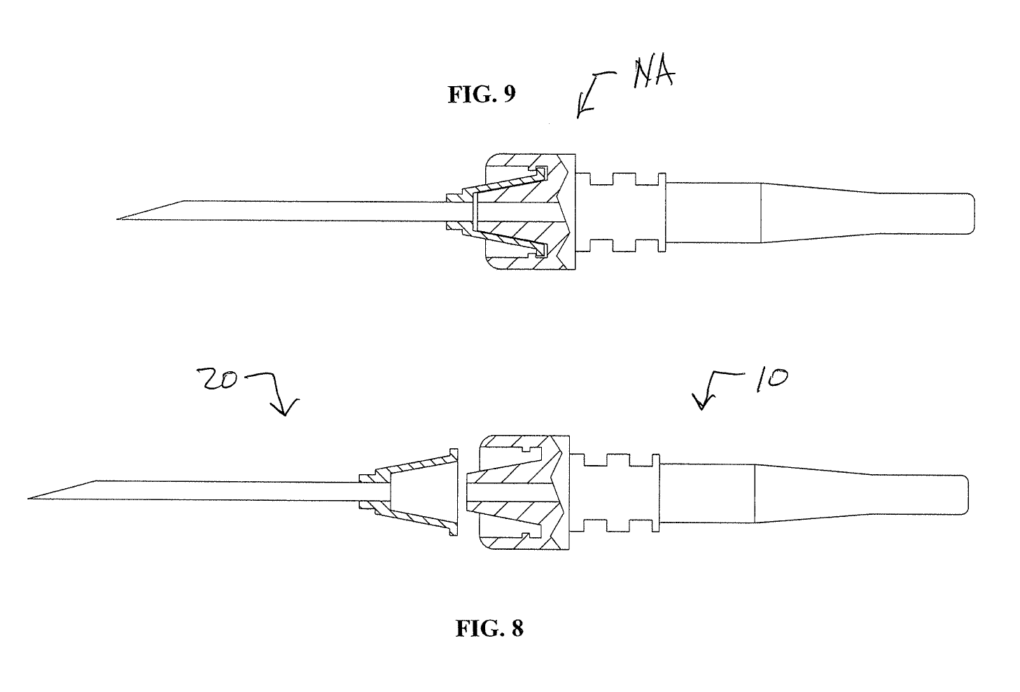

FIG. 8 shows the needle member of FIG. 5 as it is about to be connected to the needle holding member 10 of FIG. 3. This can occur, by way of non-limiting example, before the needle holding member 10 is installed on a fluid collection device or after installed therein. FIG. 9 shows a needle assembly NA resulting from the connection of the components, i.e., needle member 20 and holding member 10, shown in FIG. 8.

According to one non-limiting example, a user can connect these components while the cover 30 is installed to make for a more safe assembly of the assembly NA. Assembly of these components can also occur in a factory setting so that the assembly NA shown in FIG. 9 (but optionally additionally including at least a safety cover 30 on the forward needle) is packaged in an assembled state. In this case, a user can remove the assembly NA from its packaging and install it on a fluid collection device. In other embodiments, the needle member 20 and holding member 10 are each packaged separately and in a non-attached manner. In other embodiments, the needle member 20 and holding member 10 are each packaged separately, but with both packages attached to one another. In still other embodiments, two or more needle members 20 of the same type are packaged together. In still other embodiments, two or more needle members 20 of different types, i.e., needle sizes, are packaged together. In still other embodiments, one holding member 10 and two or more, e.g., different, needle members 20 are placed in the same package in an unassembled state, e.g., in a factory setting. This allows a user to customize the needle assembly NA and/or tailor it to the circumstances. For example, if the package contains a single holding member 10 and, e.g., three needle members of the type as shown in FIGS. 5-7, the user can place any one of the needle members 20A-20C on the holding member 10 depending on which needle member 20A-20C is called for and discard the other two. It is also possible to package a single holding member 10 along with, e.g., three individually packaged needle members of the type as shown in FIGS. 5-7. The user can place any one of the needle members 20A-20C on the holding member 10 depending on which needle member 20A-20C is called for and store the packaged and unused other two.

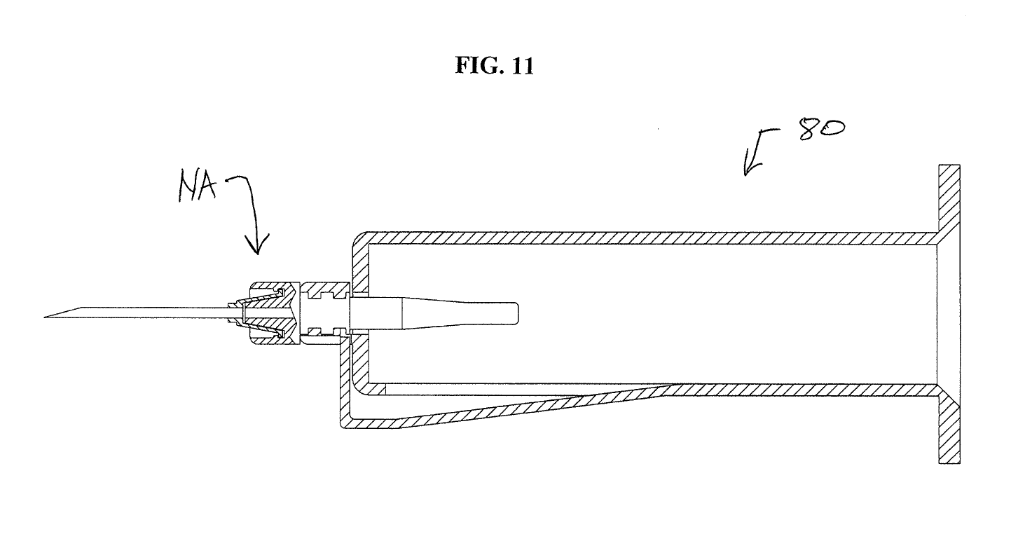

FIG. 10 shows the needle holding member 10 of FIG. 3 installed on a fluid collection device 80 in accordance with one non-limiting embodiment of the invention. In embodiments, the fluid collection device 80 can be of any of the types disclosed in U.S. 2010/0286558 to SCHRAGA. In other embodiments, the invention can also be used with any one of the fluid collection systems disclosed in U.S. 2011/0160613 to SCHRAGA. The disclosure of each of these documents is expressly incorporated by reference herein in their entireties. As details of the fluid collection devices are described in these documents, additional details of such devices are not described herein. In still other embodiments, the fluid collection device 80 can be of any of the types conventionally known provided they function with a holing member 10 having a common or standard interface. FIG. 11 shows the needle assembly NA of FIG. 9 installed on a fluid collection device 80 in accordance with one non-limiting embodiment of the invention. Again, in embodiments, the fluid collection device 80 can be of any of the types disclosed in U.S. 2010/0286558 to SCHRAGA. In other embodiments, the invention can also be used with any one of the fluid collection systems disclosed in U.S. 2011/0160613 to SCHRAGA.

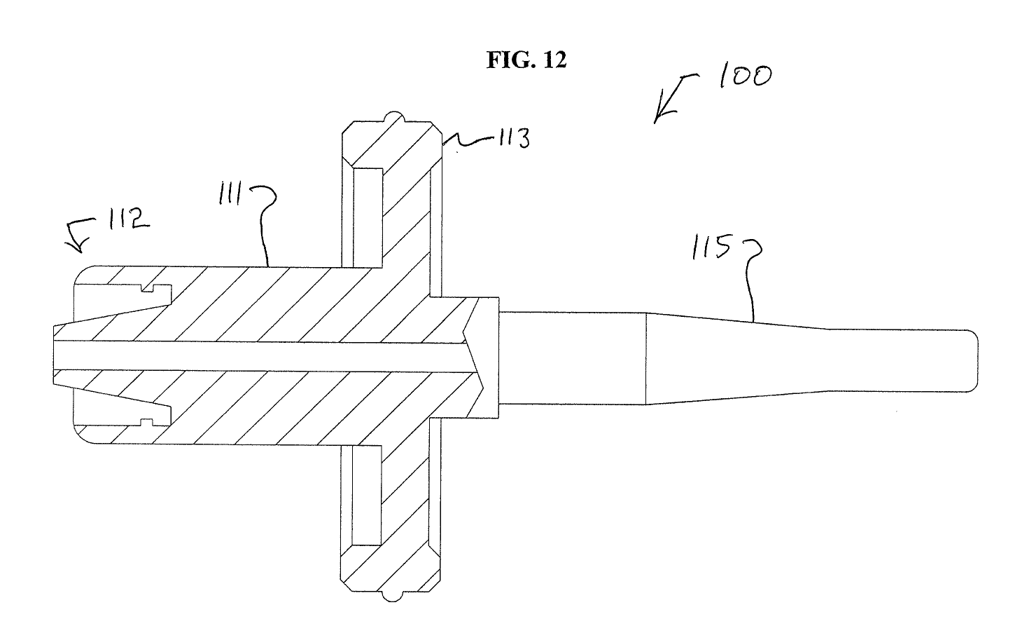

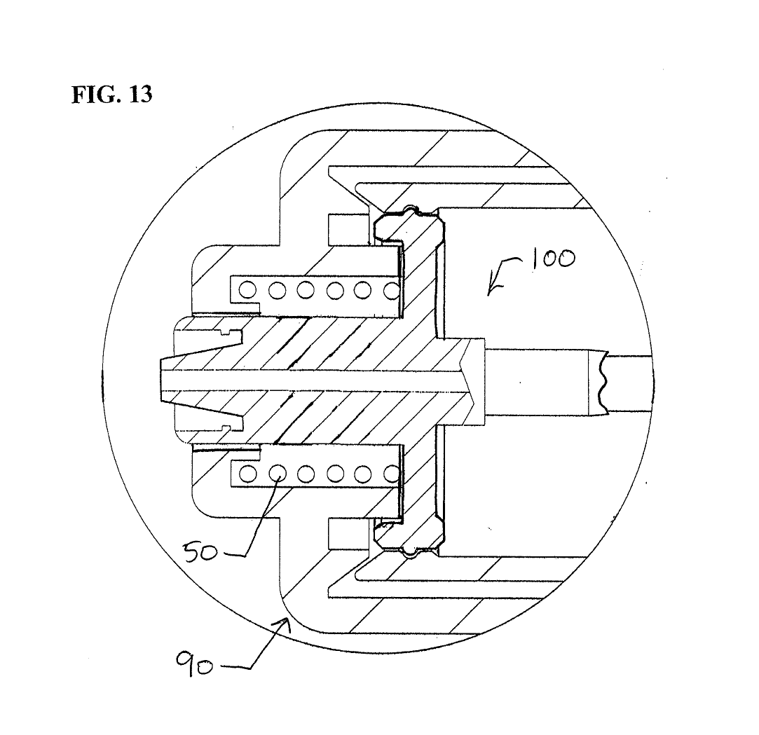

FIG. 12 shows another non-limiting embodiment of the invention. In FIG. 12, a needle holding member 100 in accordance with one non-limiting embodiment of the invention has hub portion 111, a connecting interface portion 112, a retaining section 113, an inner hollow needle, and a retractable needle cover 115. The configuration of the interface 112, i.e., the receiving portion thereof, can be of any conventional standard type interface such as a luer-lok type interface. Similarly, the configuration of elements 14 and 15 can be of any conventional type. In embodiments, the member 100 can be of any of the types disclosed in U.S. 2008/0262421 to SCHRAGA with the exception that it utilizes the interface 112 and a needle member 20 of the type described above such as that shown in FIGS. 5-7. The disclosure of U.S. 2008/0262421 is expressly incorporated by reference herein in its entirety. FIG. 13 shows one way in which a fluid collection device 90 of the type disclosed in U.S. 2008/0262421 can be modified to utilize the holding member 100 of FIG. 12. The normally compressed spring 50 is utilized to automatically cause the member 100 (typically with the needle member 20 attached) to retract into the body of the device 90 when activated after use.

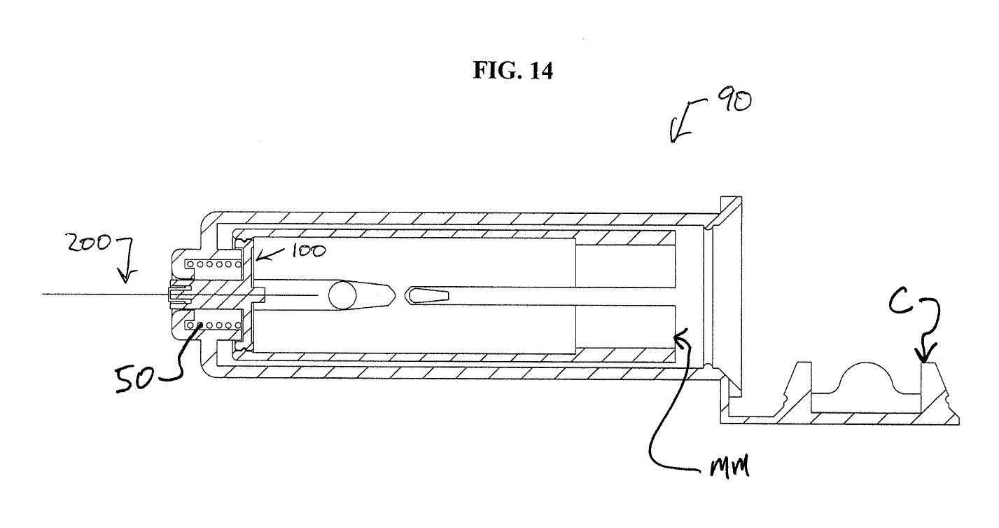

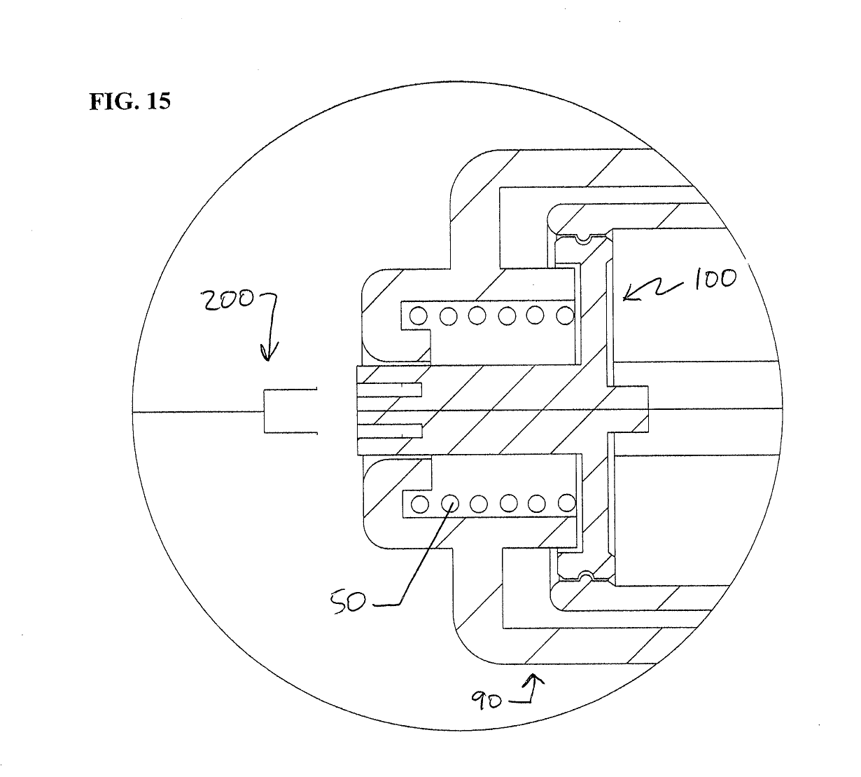

FIG. 14 shows a needle assembly installed on a fluid collection device 90 in accordance with another non-limiting embodiment of the invention. The needle assembly includes the holder member 100 and a needle member 200 shown schematically. In embodiments, the needle member 200 can be of same as the member 20 shown and described above. The fluid collection device 90 can function in a manner similar to similar devices described in U.S. 2008/0262421. For example, the device 90 can function as follows: after the device 90 is used to obtain a fluid sample, the use can move the cap C to the closed position. Upon doing so, the cap C becomes non-releasably locked and the member MM is caused to move forward slightly. This movement, in turn, results in the proximal portion of the member MM expanding radially. This radial expansion causes the holder member 100 to disengage from the member MM. At this point, the spring 50 is free to expand axially and cause the member 100 (with the needle member 200 connected thereto) to retract fully into the device 90 and be safely contained therein so that the used device 90 can be handled safely and disposed of in a safer manner. The details of how such a device (but without the member 100/200 combination) functions are disclosed in U.S. 2008/0262421 and are therefore not described in more detail herein. FIG. 15 shows the device of FIG. 14 before the (schematically shown) needle member 200 is connected to the needle holding member 100 already installed on the fluid collection device 90 in accordance with another non-limiting embodiment of the invention.

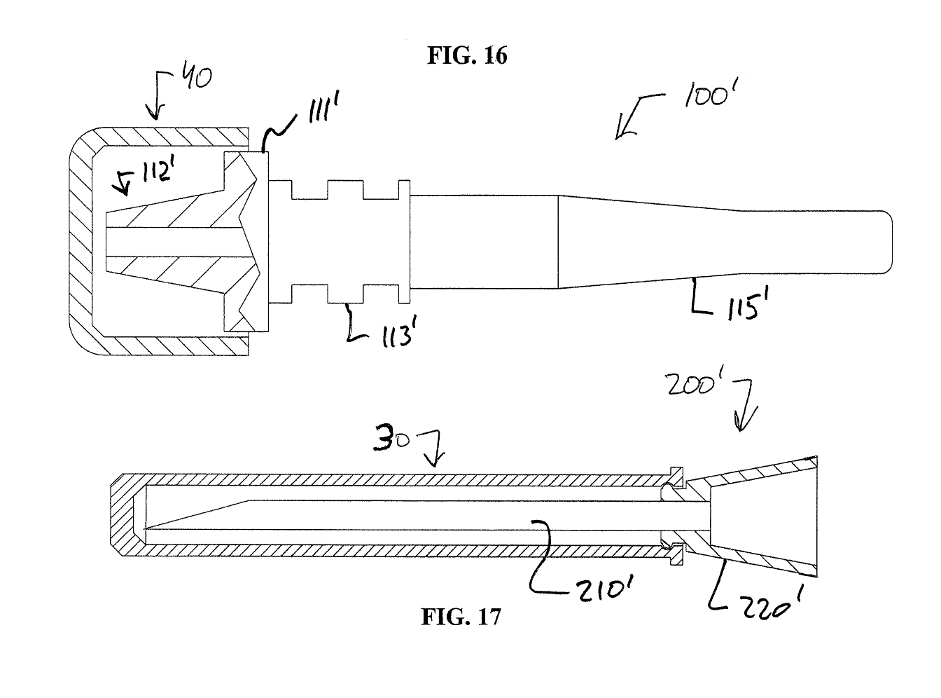

FIGS. 16 and 17 show another non-limiting embodiment of the invention. In FIG. 16, a needle holding member 100′ in accordance with another non-limiting embodiment of the invention has hub portion 111′, a connecting interface portion 112′, a retaining section 113′, an inner hollow needle, and a retractable needle cover 115′ normally covering the inner needle. The configuration of the interface 112′, i.e., the receiving portion thereof, can be of any conventional standard type interface such as a luer-lok type interface without the locking outer portion. Similarly, the configuration of elements 113′ and 115′ can be of any conventional type. An optional removable 40 can be used to protect the interface 112′.

In FIG. 17, a needle member 200′ in accordance with another non-limiting embodiment of the invention is shown in a prior-use configuration. In this configuration, a removable needle cover 30 is shown mounted covering the hollow needle portion 210′ of the needle member 200′. The cover 30 has a closed proximal end and an open distal end which is removably and releasably connected (via e.g., a snap and/or projection/recess connection) to the connecting interface section 220′ of the member 200′.

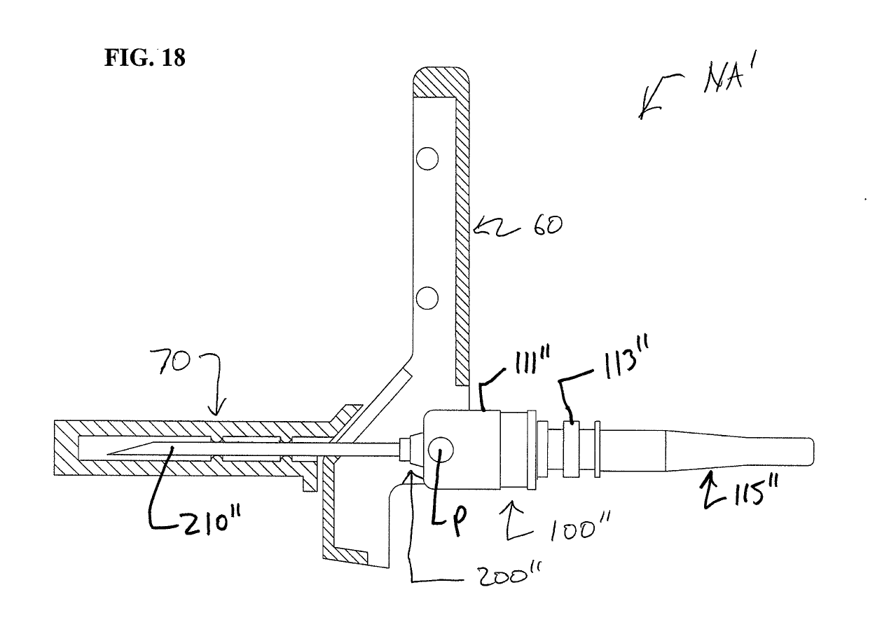

FIG. 18 shows another non-limiting embodiment of the invention. In FIG. 18, a needle holding member 100″ in accordance with another non-limiting embodiment of the invention has hub portion 111″, a connecting interface portion, a retaining section 113″, an inner hollow needle, and a retractable needle cover 115″ normally covering the inner needle. The configuration of the interface, i.e., the receiving portion thereof, can be similar to that of FIG. 1 and/or of any conventional standard type interface such as a luer-lok type interface. In addition, the member 100″ includes two oppositely arranged projections P arranged on the section 111″ to which is pivotally mounted a needle shield 60. An optional safety cover 70 covers the needle 210″ of the removably mounted needle member 200″. The details of how such a device (but without the removable connection between members 100″/200″) functions are disclosed in U.S. Ser. No. 13/458,468 filed on Apr. 27, 2012 to SCHRAGA and are therefore not described in more detail herein. The disclosure of U.S. Ser. No. 13/458,468 filed on Apr. 27, 2012 is expressly incorporated by reference herein in its entirety. In embodiments, the assembly NA′ shown in FIG. 18 is packaged for use in the configuration shown. To use the assembly, a user need only install the assembly on a fluid collection device of the type shown in, e.g., FIG. 11, and then remove the cover 70. After use, the user can flip up the shield 60 so that it non-removably covers the needle 210″. In this position, the user is prevented from removing the needle member 200″ from the holding member 100″.

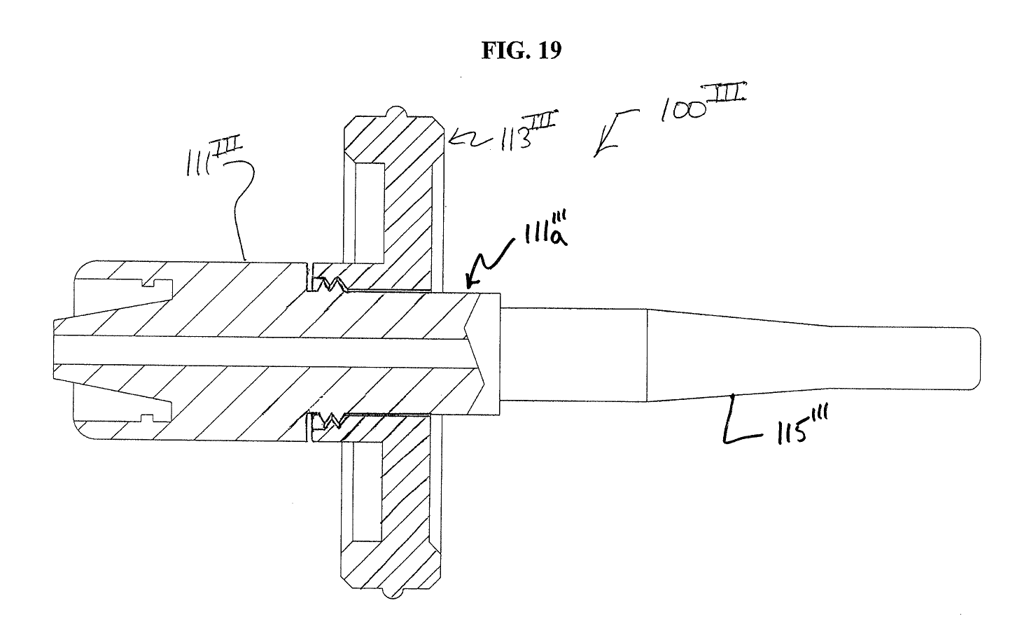

FIG. 19 shows another non-limiting embodiment of the invention. In FIG. 19, a needle holding member 100^{III} in accordance with another non-limiting embodiment of the invention has hub portion 111^{III}, a connecting interface portion, a retaining section 113^{III} threadably and/or removably installable and/or installable only once on the section 100a^{III}, an inner hollow needle, and a retractable needle cover 115^{III} normally covering the inner needle. The configuration of the interface, i.e., the receiving portion thereof, can be similar to that of FIG. 1 and/or of any conventional standard type interface such as a luer-lok type interface. In embodiments, the assembly shown in FIG. 19 is packaged for use in the configuration shown. To use the assembly, a user need only install the assembly on a fluid collection device of the type shown in, e.g., FIG. 14. The user can then install one of the needle members of the type shown in FIGS. 5-7. After use, both the needle member and the assembly 100^{III} are safely arranged within the body shown I FIG. 14 and can be safely disposed of.

The devices described herein can also utilize one or more features disclosed in the documents expressly incorporated by reference herein. Furthermore, one or more of the various parts of the device can preferably be made as one-piece structures by e.g., injection molding, when doing so reduces costs of manufacture. Non-limiting materials for most of the parts include synthetic resins such as those approved for syringes, blood collection devices, or other medical devices. Furthermore, the invention also contemplates that any or all disclosed features of one embodiment may be used on other disclosed embodiments, to the extent such modifications function for their intended purpose.

It is noted that the foregoing examples have been provided merely for the purpose of explanation and are in no way to be construed as limiting of the present invention. While the present invention has been described with reference to an exemplary embodiment, it is understood that the words which have been used herein are words of description and illustration, rather than words of limitation. Changes may be made, within the purview of the appended claims, as presently stated and as amended, without departing from the scope and spirit of the present invention in its aspects. Although the present invention has been described herein with reference to particular means, materials and embodiments, the present invention is not intended to be limited to the particulars disclosed herein; rather, the present invention extends to all functionally equivalent structures, methods and uses, such as are within the scope of the appended claims.

What is claimed:

1. A fluid collection device comprising:
a body having a front end and a back end;
the back end that is at least one of open and sized and configured to receive therein a receptacle;
a needle holding member arranged in an area of the front end of the body; and
a needle holding member comprising a needle connecting interface adapted to mate with a needle interface

2. The fluid collection device of claim 1, wherein the needle holding member is a spring biased member that is movable from an initial position to a retracted position within the body.

3. The fluid collection device of claim 1, wherein the needle connecting interface is a luer-lock interface.

4. The fluid collection device of claim 1, further comprising a spring structured and arranged to move the needle holding member to a retracted position within the body when a movable member arranged with the body experiences at least one of:
a front end of the movable member is caused to expand generally radially;
a back end of the movable member is caused to contract generally radially;
a front end of the movable member is caused to expand generally radially when a back end of the movable member is caused to contract generally radially;
axial movement caused by a cap closing off the back end of the body, wherein, during the closing off of the back end of the body, a tapered surface of a back end of the movable member is contacted by a portion of the cap;
circumferential portions of a front end of the movable member are caused to expand outwardly or generally radially so as to allow the needle holding member to disengage from the front end of the movable member; and
portions of a front end of the movable member separated by slots are caused to expand outwardly or generally radially so as to allow the needle holding member to disengage from the front end of the movable member.

5. A fluid collection/injection device comprising:
a body having a front end, a back end, and a main hollow section arranged between the front and back ends;
a needle hub securing section arranged on the front end and being structured and arranged to receive therein a needle member;
the needle hub securing section comprises a fixed part and a movable part;
a needle holding member ,connectable to the needle hub securing section; and
the needle holding member comprising a needle connecting interface adapted to mate with a needle interface,
wherein the fixed part is integrally formed with the front end and the movable part is arranged on a member that has one end which is one of:
fixed to a portion of the main hollow section;
connected to a portion of the main hollow section via a living hinge;
removably connected to a portion of the main hollow section; and
integrally formed with the main hollow section.

6. The device of claim 5, wherein the body is one of generally cylindrical and generally tubular.

7. The device of claim 5, wherein the fixed part and the movable part form a generally cylindrical section which the movable part is in an initial position.

8. The device of claim 5, wherein the fixed part and the movable part each generally comprise one-half of an internal locking thread structured and arranged to engage with an external thread of the needle member.

9. The device of claim 5, wherein the member has one end which is fixed to the portion of the main hollow section.

10. The device of claim 5, wherein the member has one end which is removably connected to the portion of the main hollow section.

11. A retractable medical device comprising:
a body having and a back end that is at least one of open and sized and configured to receive therein a receptacle;
a movable member arranged within the body, the movable member having a back end and a front end;
a movable holding member arranged in an area of the front end of the movable member;
a needle connecting interface adapted to mate with a needle interface and being movable with the movable holding member; and
a spring structured and arranged to move the movable holding member to a retracted position within the movable member when the movable member experiences at least one of:
the front end of the movable member is caused to expand generally radially;
the back end of the movable member is caused to contract generally radially;
the front end of the movable member is caused to expand generally radially when the back end of the movable member is caused to contract generally radially;
axial movement caused by a cap closing off the back end of the body, wherein, during the closing off of the back end of the body, a tapered surface of the back end of the movable member is contacted by a portion of the cap;
circumferential portions of the front end of the movable member are caused to expand outwardly or generally radially so as to allow the needle holding member to disengage from the front end of the movable member; and
portions of the front end of the movable member separated by slots are caused to expand outwardly or generally radially so as to allow the needle holding member to disengage from the front end of the movable member.

12. The device of claim 11, wherein the needle connecting interface is a luer-lock interface.

13. A fluid collection/injection device comprising:
a body having a front end, a back end, and a main hollow section arranged between the front and back ends;
a needle hub securing section arranged on the front end;
a needle receiving member having a needle connecting interface; and
the fluid collection/injection device is structured and arranged to utilize at least an operational mode, an operational mode, and a post-use mode;
in the installation mode, the needle receiving member being coupled to the body via the needle hub securing section;
in the operational mode, fluid passing through the needle receiving member and into or out of a receptacle inserted into the main hollow section; and
in the post-use mode, a safety cover at least one of;
prevents re-use of the fluid collection/injection device;
prevents removal of the needle receiving member from the fluid collection/injection device;
prevents removal of the needle receiving member from the needle hub securing section;
locks in a covering position and prevents removal of a needle from the needle receiving member;
activates release of the needle receiving member from the body such that when the safety cover is in a position covering a needle of the needle receiving member, the needle receiving member can fall out of the needle hub securing section;
activates release of the needle receiving member from the body such that when the safety cover is in a position covering a needle of the needle receiving member, the needle receiving member is no longer securely retained to the needle hub securing section;
releases a securing engagement between the needle receiving member and the needle hub securing section;
unlocks a locking connection between the needle receiving member and the needle hub securing section; and
moves a mechanism arranged in an area of the needle hub securing section which releases a connection between the needle receiving member and the needle hub securing section.

14. The device of claim 13, wherein the needle connecting interface is a luer-lock interface.

15. A method drawing fluid using the device of claim 1, the method comprising:
installing the needle connecting interface; and
thereafter installing a needle member having a standard interface on the needle connecting interface.

16. A method drawing fluid using the device of claim 1, the method comprising:
installing a needle member having a standard interface on the needle connecting interface of the needle holding member so as to form a needle assembly; and
thereafter installing the needle assembly on the body.

17. A method drawing fluid using the device of claim 1, the method comprising:
installing a needle member having a standard interface on the needle connecting interface;
one of:
injecting fluid through the needle; and
withdrawing fluid into a container installed in the body via a needle of the needle member.

18. A needle assembly for a fluid collection device comprising:
a needle holding member installable on a fluid collection device;
a needle connecting interface arranged on one end of the needle holding member; and
a needle member having a needle interface,
wherein the needle member is at least one of:
installable on the needle holding member such that a needle of the needle member is in fluid communication with another needle arranged on the needle holding member; and
removably installable on the needle holding member.

19. A double-ended needle assembly comprising:
a needle holding member installable on a fluid collection device and comprising a needle connecting interface arranged on one end of the needle holding member and a needle arranged on another end; and
a needle member having a standard needle interface compatible with the needle connecting interface of the needle holding member.

20. The assembly of claim 19, wherein the needle member is at least one of:
installable on the needle holding member such that the needle of the needle member is in fluid communication with the needle arranged on the needle holding member; and
removably installable on the needle holding member.
